(19) United States
(12) Patent Application Publication
(10) Pub. No.: US 2007/0118041 A1
(43) Pub. Date: May 24, 2007

(54) APPARATUS AND METHOD OF HEART FUNCTION ANALYSIS

(75) Inventors: Masahide Nishiura, Minato-ku, Tokyo (JP); Yasuhiko Abe, Otawara-shi, Tochigi (JP); Tetsuya Kawagishi, Otawara-shi, Tochigi (JP); Ryoichi Kanda, Otawara-shi, Tochigi (JP)

Correspondence Address: AMIN, TUROCY & CALVIN, LLP, 1900 EAST 9TH STREET, NATIONAL CITY CENTER, 24TH FLOOR,, CLEVELAND, OH, 44114, US

(73) Assignees: KABUSHIKI KAISHA TOSHIBA, Tokyo (JP); TOSHIBA MEDICAL SYSTEMS CORPORATION, Otawara-shi (JP)

(21) Appl. No.: 11/554,913

(22) Filed: Oct. 31, 2006

(30) Foreign Application Priority Data
Oct. 31, 2005 (JP) 2005-316853

(51) Int. Cl. A61B 5/02

(52) U.S. Cl. 600/508

(57) ABSTRACT

A heart function analysis apparatus adapted to analyze a motion of a heart by using time-series image data representing an image of the heart. The heart function analysis apparatus includes a strain-value acquisition unit configured to acquire time-series strain values in thickness of a heart muscle from the time-series image data, a normalized strain value calculation unit configured to normalize the time-series strain values, and an output unit configured to output the time-series normalized strain values.

### CROSS-REFERENCE TO RELATED APPLICATIONS

This application is based upon and claims the benefit of priority from the prior Japanese Patent Application No. 2005-316853, filed on Oct. 31, 2005; the entire contents of which are incorporated herein by reference.

### TECHNICAL FIELD

The present invention relates to a heart function analysis apparatus and method and, more particularly, to a heart function analysis apparatus and method enabled to output time-series data representing strain values in a form suitable for diagnosis.

### BACKGROUND OF THE INVENTION

A heart function analysis apparatus adapted to measure and analyze cardiac motion from time-series image data representing an image of a heart has been developed to diagnose cardiac diseases, such as a cardiac infraction and a cardiac angina, in detail.

Japanese Patent Application Laid-Open (KOKAI) No. 2003-325521 describes a related system adapted to calculate a heart function evaluation value according to temporal change in size of a left ventricle lumen of a heart. In the case of a cross-sectional image of the heart, the size of the left ventricle lumen is represented by the cross-sectional area thereof. The cross-sectional area of the left ventricle lumen is obtained by first determining an endocardial border (that is, an inner border of a heart-muscle wall) and then calculating the cross-sectional area thereof according to the shape of the endocardial border. That is, the temporal change in size of the left ventricle lumen is calculated from change in position of the endocardial border.

However, sometimes, a method of evaluating a heart function according to change in the endocardial border does not clearly reflect abnormality of a heart muscle. For example, in a case where decrease in function of expanding and contracting heart muscles locally occurs, a decrease in motion of the endocardial border of an abnormal part pulled by the movement of neighboring normal heart muscles does not clearly occur. Consequently, in such a case, a clear difference in the evaluation value calculated from change in the endocardial border does not occur.

In the case of a related method adapted to evaluate the heart function by dividing the heart muscle into local parts, a central point for calculating the cross-sectional area of each of parts, into which the lumen is divided, is needed. In a case where the central point is fixed, the parallel translation of the entire heart affects the cross-sectional area of each of the parts, into which the lumen is divided. Thus, to prevent the cross-sectional area of each of the parts from being affected by the translation of the entire heart, it becomes necessary to determine the central point in response to the translation of the entire heart.

As described above, the related system has a problem in that the heart function evaluation value does not clearly reflect abnormality of a heart muscle.

Also, the related method adapted to evaluate the heart function by dividing the heart muscle into local parts has a problem in that it is necessary to determine the central point so that the cross-sectional area of each of the parts from being affected by the movement of the entire heart.

The present invention is accomplished to solve the aforementioned problems. Accordingly, the present invention provides a heart function analysis apparatus and method that is enabled to obtain an evaluation value which clearly affects abnormality of a heart function, and that does not require the setting of the central point in the case where the heart function is evaluated by dividing the heart muscle into parts.

### BRIEF SUMMARY OF THE INVENTION

According to an aspect of the present invention, there is provided a heart function analysis apparatus adapted to analyze a motion of a heart by using time-series image data representing an image of the heart. This heart function analysis apparatus includes a strain-value acquisition unit configured to acquire time-series strain values corresponding to a part of the heart according to the time-series image data, a normalization unit configured to calculate time-series normalized strain values by normalizing the time-series strain values, and an output unit configured to output the time-series normalized strain values.

The heart function analysis apparatus according to the invention can clearly reflect abnormality of the heart function by calculating temporal change in the normalized strain value. Also, the heart function analysis apparatus according to the invention does not require the setting of the central point in the case where the heart function is evaluated by dividing the heart muscle into parts.

### BRIEF DESCRIPTION OF THE DRAWINGS

FIG. 1 is a block diagram illustrating the configuration of a heart function analysis apparatus 10 according to an embodiment of the invention.

FIG. 2 is a flowchart illustrating an operation of this embodiment.

FIG. 3 is a view illustrating the detection of displacement between frames at the position of a tracking point.

FIG. 4 is a view illustrating a method of specifying a measurement object.

FIG. 5 is a view illustrating the tracking of endpoints of the measurement object.

FIG. 6 is a schematic view illustrating the arrangement of measurement objects in the case of acquiring strain values over the entire heart muscle.

FIG. 7A is a graph illustrating examples of temporal changes of strain values.

FIG. 7B is a graph illustrating examples of temporal changes of normalized strain values.

FIG. 8 is a graph illustrating a temporal change of a normalized strain value.

FIG. 9 is a view illustrating an example of color display of parts of a heart muscle, which respectively correspond to time periods required to normalized strain values.

FIG. 10 is a graph illustrating a standard change pattern and a measured change pattern.

### DETAILED DESCRIPTION OF THE INVENTION

Hereinafter, a heart function analysis apparatus 10 according to an embodiment of the invention is described below with reference to the accompanying drawings.

(1) Configuration of Heart Function Analysis Apparatus 10

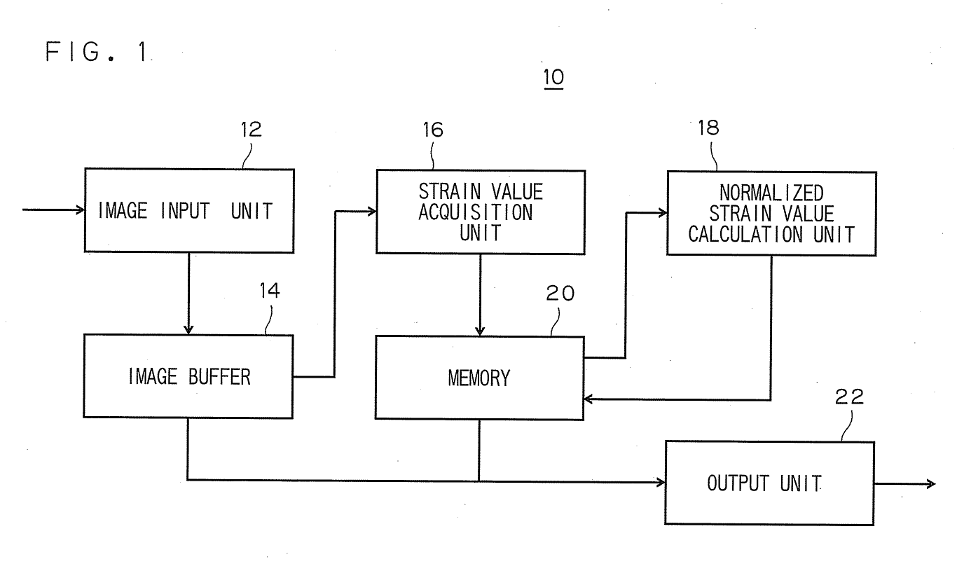

FIG. 1 is a block diagram illustrating the configuration of the heart function analysis apparatus 10 according to the present embodiment of the invention.

The heart function analysis apparatus 10 has an image input unit 12 adapted to input time-series image data, an image buffer 14 adapted to store image data, a strain value acquisition unit 16 adapted to obtain time-series data representing strain values of a heart, from image data, a normalized strain value calculation unit 18 adapted to normalize time-series strain value data, a memory 20 adapted to store data representing the strain value and the normalized strain value, and an output unit 22 adapted to output time-series data representing normalized strain values.

The heart function analysis apparatus 10 can be implemented by using, for example, a general-purpose computer apparatus 10 as basic hardware. That is, the image input unit 12, the strain value acquisition unit 16, the normalized strain value calculation unit 18, and the output unit 22 can be implemented by causing a processor mounted in the computer to execute a program. In this case, the heart function analysis apparatus 10 may be implemented by preliminarily installing the program in the computer apparatus. Alternatively, the program may be either stored in a storage media, such as a CD-ROM, or distributed through a network. Then, the heart function analysis apparatus 10 may be implemented by installing the program in the computer apparatus. Also, the image buffer 14 and the memory 20 can be implemented by appropriately utilizing storage media, such as a memory 20 incorporated in the computer or provided outside of the computer, a hard disk, a CD-R, a CD-RW, a DVD-RAM, or a DVD-R.

(2) Operation of Heart Function Analysis Apparatus 10

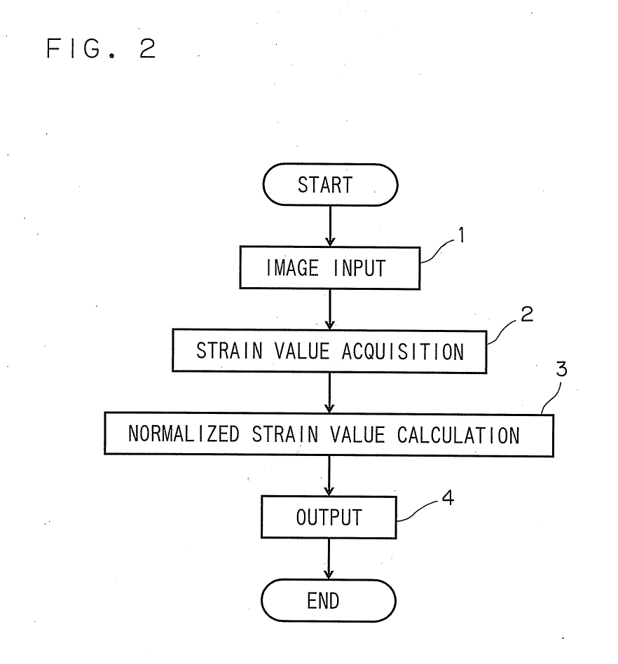

Next, an operation of the heart function analysis apparatus 10 is described below by referring to a flowchart shown in FIG. 2.

(2-1) Image Input Unit 12

First, in step 1, time-series image data is inputted from the image input unit 12. The time-series image data may be inputted from a medical image diagnosis apparatus, such as an ultrasonic diagnosis apparatus, an X-ray CT scanner, or an MRI machine. Alternatively, image data stored in an image server may be used as input data. The inputted image data is stored in the image buffer 14.

(2-2) Strain Value Acquisition Unit 16

In step 2, the strain value acquisition unit 16 acquires time-series strain values corresponding to the heart from the time-series image data by, for example, the following method.

(2-2-1) First Acquisition Method

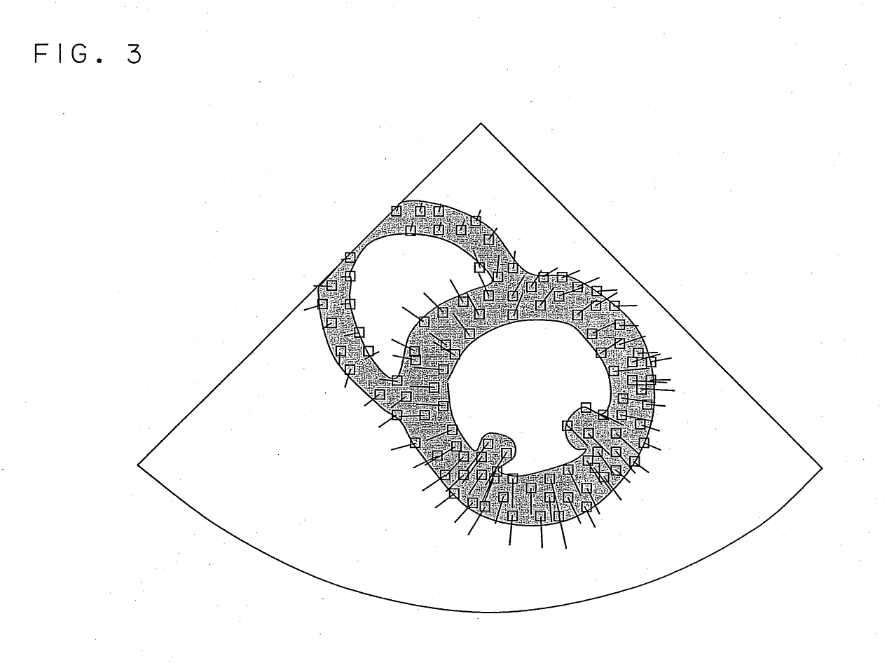

A first method of acquiring times-series strain values is described below with reference to FIGS. 3 to 6.

The first method is performed by performing the following image analysis procedure.

According to the image analysis procedure for acquiring strain values, first, tracking points are disposed in an image. It is advisable to select positions of feature points, such as brightness peaks and corners, of an image pattern as the tracking points.

Subsequently, an image pattern formed around the tracking point, whose position is a reference position, is used as a template image. Thus, template matching is performed between this current frame image and the subsequent frame image. In the template matching, the position of a part of the next frame image, which is most analogous to the template image, is searched for. Consequently, the position of a destination in the subsequent image frame, to which each of the tracking points in the current frame is displaced, is detected (see FIG. 3).

Thus, the process of disposing the tracking points and performing the template matching is performed on adjacent frames, for example, first and second frames, and second and third frames. Consequently, the movement of each of the tracking points in time-series images is detected.

Subsequently, a strain value S is calculated according to the following equation (1). That is, the strain value S is defined to be a value obtained by dividing a change in length of a measurement object by a length at a reference time as follows.
S=(L−L_{ref})/L_{ref} (1)

In a case where a wall thickness of the heart is a measurement object, a strain value WT is expressed by the following equation (2):
WT=(Lₜ−L_{ref})/L_{ref} (2)
where L_{ref} is a wall thickness at the reference time, and Lₜ is a wall thickness at a moment t.

In this case, the strain value WT is also referred to as a wall-thickness increasing rate or a wall-thickness changing rate.

The strain value may be expressed in percentage by multiplying the value WT by 100.

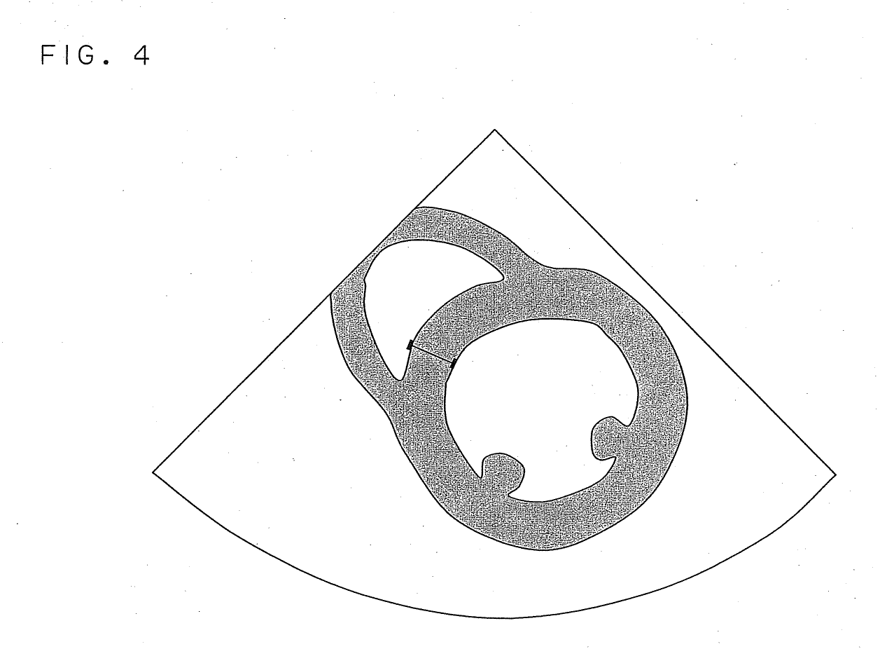

To measure the strain value, in the present embodiment, first, a reference frame is designated. Then, a measurement object is specified in this frame. It is advisable to specify the measurement object by designating the positions of both endpoints of the measurement object (see FIG. 4). It is recommendable to designate the positions of both endpoints by using pointing devices, such as a mouse. Alternatively, the positions of both endpoints may be set automatically or semi-automatically by a method as will be described later.

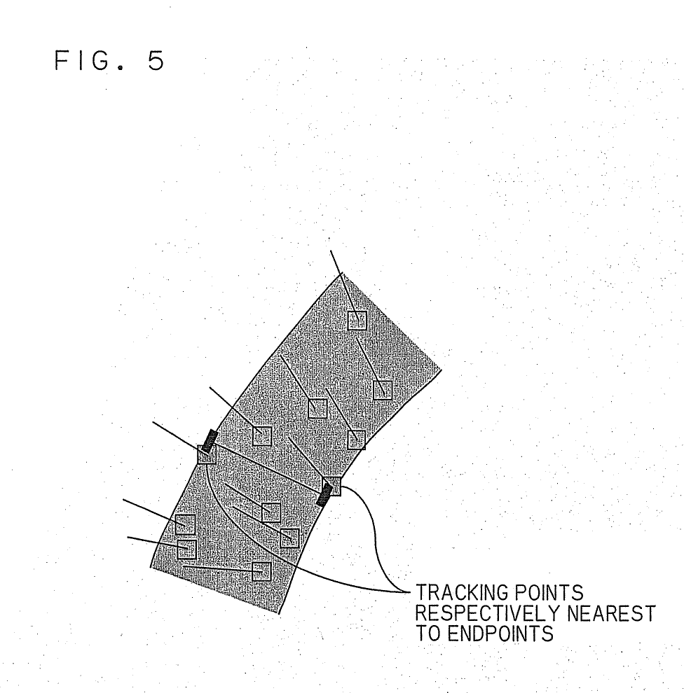

After the positions of both endpoints are designated, the positions of destinations in the subsequent frame, to which the endpoints are displaced, are estimated. The estimation of the positions of the destinations is performed by using information on the movement of the endpoints, which is obtained by the template matching. For example, a method of approximating the movement of each endpoint by that of a tracking point nearest thereto can be used (see FIG. 5). Alternatively, the movement of each endpoint may be estimated by interpolating the movements of a plurality of tracking points close thereto. A linear interpolation method and a two-dimensional spline may be used as the method of interpolating the movements.

Thus, the positions of both the endpoints of the measurement object can be tracked over time-series frames. Consequently, the distance between both the endpoints can be calculated corresponding to each frame. The strain value is calculated according to the equation (1) or (2) from the calculated distance between both the endpoints.

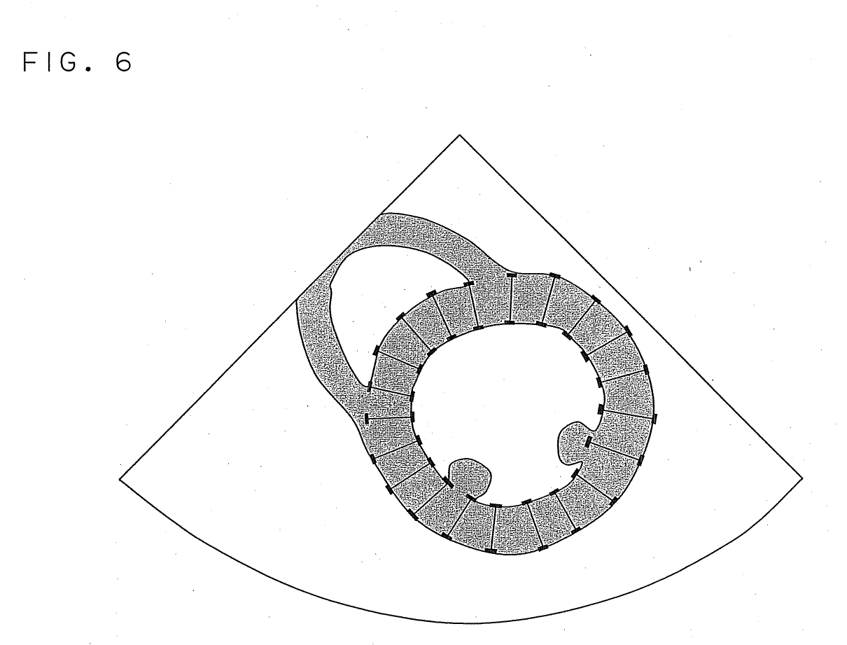

In the case of the presence of a plurality of measurement objects, both endpoints of each of the plurality of measurement objects are set. Then, the aforementioned procedure for calculating the strain value is performed on each of the measurement objects. In a case where a heart is to be analyzed, it is convenient for understanding the distribution of the strain values of a heart muscle to set a plurality of measurement objects on the heart muscle. The strain values can be calculated over the entire heart muscle by disposing a plurality of both endpoints on inner and outer sides of the heart muscle, as shown in FIG. 6. To simply arrange both endpoints as shown in FIG. 6, it is advisable to extract or designate, for example, the inner and outer contours of the heart muscle and to then dispose the endpoints on the contours.

(2-2-2) Second Acquisition Method

A second strain value acquisition method may be adapted so that the destinations of both endpoints of a measurement object in the subsequent frame are calculated by using tissue speed information obtained in a Doppler mode of an ultrasound, and that then, the strain value is computed.

According to this method, ultrasonic pulses are emitted a plurality of times. Then, the movement speed of the object is detected from the difference in delay time among reflection pulses. In a case where the object moves, the speed thereof can be detected due to time-lags among a plurality of reflection pulses. Information on the detected speed includes information on an ultrasonic-beam-direction component of the speed. Thus, an actual speed can be estimated by additionally supposing a movement direction or by manually inputting information on the movement direction. Thus, the position of the endocardial border is tracked by using the speed information obtained in this way. Consequently, the strain value can be calculated.

(2-2-3) Third Acquisition Method

A third acquisition method may be adapted so that the strain values are separately calculated, and that only data representing the calculated strain values is inputted to the apparatus.

For example, the endocardial border is tracked by manually designating the inner and outer border positions of a heart muscle. The strain value can be calculated from change in thickness of the heart muscle.

According to the aforementioned procedures, the strain values at one or more parts of the heart can be calculated corresponding to each frame.

The calculated strain values are stored in the memory 20.

(2-3) Normalized Strain Value Calculation Unit 18

Subsequently, in step 3, the normalized strain value calculation unit 18 normalizes the time-series strain values calculated respectively corresponding to frames.

First, the manner of ordinary change in strain value in the direction of thickness of a heart muscle is described below. The thickness of the heart muscle increases as a cardiac ventricle contracts since the ventricle expands to a maximum size. Thus, the strain value increases. The thickness of the heart muscle decreases when the cardiac ventricle enters an expansion stage after contracted. Thus, the strain value decreases and almost returns to an initial strain value.

Hereinafter, normalization methods are described.

(2-3-1) First Normalization Method

According to a first normalization method, a time interval, in which the evaluation is performed, is separately determined. Then, a normalized strain value NS₁ is defined by a ratio of a difference between a current strain value and a minimum strain value of a measurement object to a difference between a maximum fluctuation range of the strain value thereof, as expressed by the following equation (3):
NS₁=(Sₜ−Sₘᵢₙ)/(Sₘₐₓ−Sₘᵢₙ) (3)
where Sₜ is a strain value at a moment t, and Sₘₐₓ and Sₘᵢₙ are the maximum value and the minimum value of the strain value of the measurement object, respectively. In a case where the minimum value of the strain value is 0, for example, in a case where a time phase, in which the strain value has the minimum value, is set to be a reference one, the strain value NS₂ is expressed by the following equation (4):
NS₂=Sₜ/Sₘₐₓ (4)

According to this definition, the normalized strain value is expressed as a rate of the strain value to the maximum fluctuation range thereof, instead of the strain value itself.

Even in a case a heart normally operates, it is assumed that the maximum strain value varies with parts of a heart muscle. Thus, in a case where the strain values of a plurality of different parts of the heart muscle are simultaneously evaluated, the evaluation can be achieved by using a same measure, without being affected by the value. This method is effective, especially, in a case where the gradient of temporal change of a strain value is evaluated.

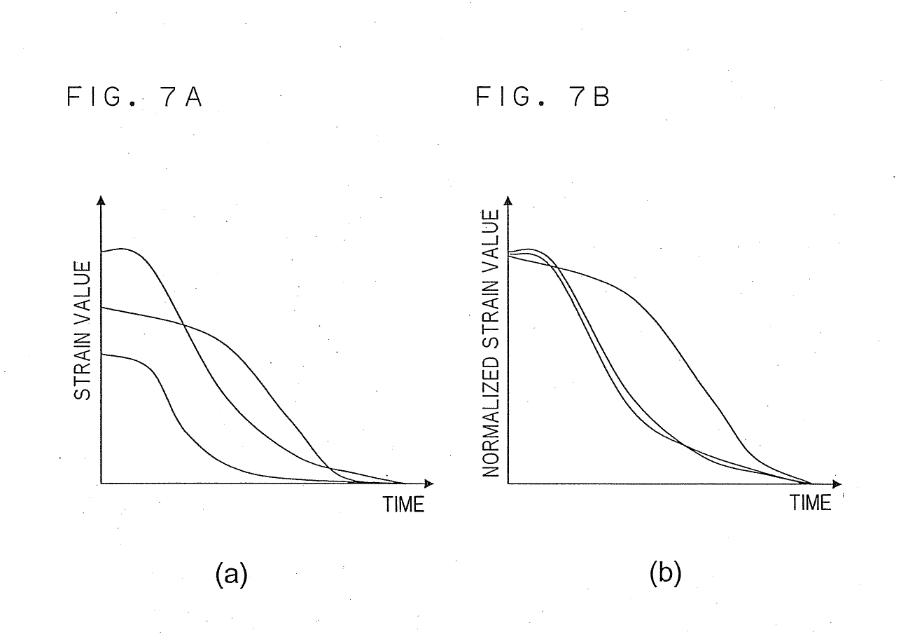

FIGS. 7A and 7B schematically illustrate this fact. FIG. 7A is an example of a graph illustrating temporal changes of strain values themselves. As is understood from FIG. 7A, it is difficult to determine according to comparison among the curves representing the strain values whether the strain values change rapidly or slowly. In contrast, FIG. 7B is an example of a graph illustrating temporal changes of normalized strain values. As is apparent from FIG. 7B, data representing the normalized strain value, whose temporal change is slow, can easily be determined, regardless of the magnitude of the maximum value of the strain value.

(2-3-2) Second Normalization Method

According to a second normalization method, a time interval, in which the evaluation is performed, is separately determined. Then, a normalized strain value NS₃ is defined by a ratio of a difference between a current strain value and an initial strain value to a difference between a difference between a terminal strain value and the initial strain value, as expressed by the following equation (5):
NS₃=(Sₜ−Sₛₜₐᵣₜ)/(S_{end}−Sₛₜₐᵣₜ) (5)
where Sₜ, Sₛₜₐᵣₜ and S_{end} are a strain value at a moment t, an initial strain value at the start of the time interval, a terminal strain value at the end of the time interval.

Thus, according to this method, in a case where the variation range of the strain value over the entire time interval is set to be 100%, the apparatus can obtain information on what percent of the variation range of the strain value changes from the start to a middle point in the time interval. Consequently, this method is effective in understanding whether the temporal change of the strain value in a systole time, a diastole time, or an optional time interval is drastic or slow.

The normalized strain values NS calculated in this way are stored in the memory 20.

(2-4) Output Unit 22

Finally, in step 4, the output unit 22 outputs the normalized strain value.

It is advisable to configure the output unit 22 to perform at least one of the display of a graph illustrating change of a normalized strain value, the display of a normalized strain value at a moment at which a designated time has elapsed from the start of a set time interval, and the display of colors respectively corresponding to normalized strain values, which are superposed at the position of a measurement object on an image.

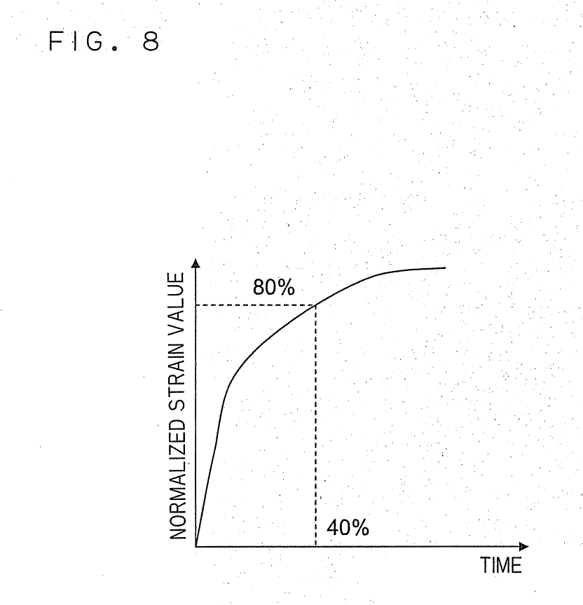

FIG. 8 shows an example of the graph illustrating change of a normalized strain value. Abscissas represent time. Ordinates represent a normalized strain value. Thus, FIG. 8 shows temporal change of the normalized strain value. Also, a normalized strain value (80% in the case of the example shown in FIG. 8) at a moment, at which a designated time (40% of a set time interval in the case of the example shown in FIG. 8) has elapsed from the start of the set time interval, is indicated in the graph. This is effective in comparing the normalized strain values with one another in the set time interval.

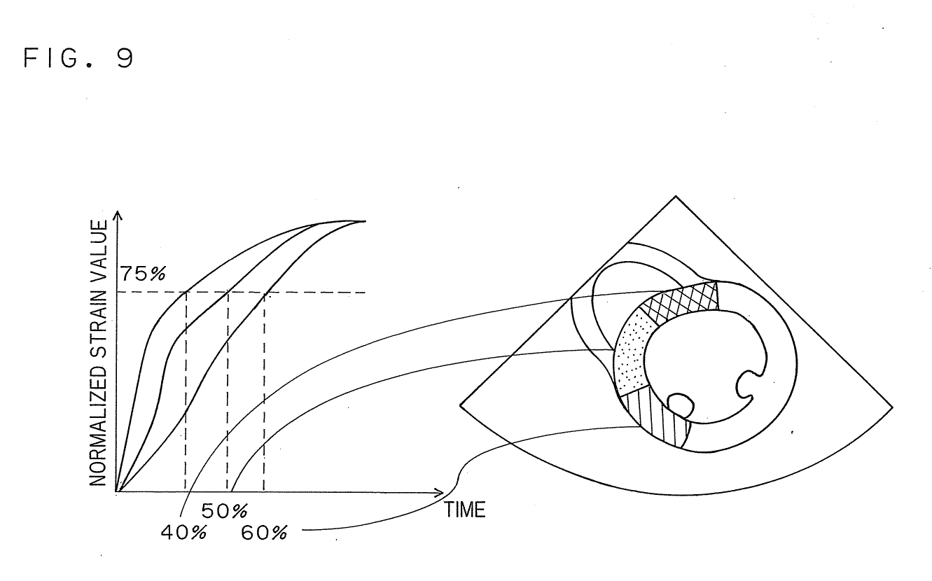

Also, as illustrated in FIG. 9, a part of the heart is displayed in color in an image of the heart according to a reach time at which the normalized strain value of the part of the heart reaches a predetermined value. Thus, the relation between the change of the normalized strain value of a part and the position of the part can easily be understood. This color display of the parts of the heart may be performed according to the normalized strain value at the part at a moment at which the designated time has elapsed. In FIG. 9, the displays of the parts in different colors are indicated by different patterns, respectively.

The aforementioned displays performed by the output unit 22 facilitate understanding of the differences in the normalized strain value among the parts of the heart muscle and the differences in the temporal change of the normalized strain value thereamong.

(3) Modifications

The present invention is not limited to the aforementioned embodiments. In a stage of carrying out the present invention, the invention can be implemented by modifying constituent elements without departing from the spirit and scope of the present invention. Also, various inventions can be made by appropriate combinations of a plurality of constituent elements disclosed in the description of the embodiments. For example, several constituent elements may be eliminated from the overall constituent elements disclosed in the description of the embodiments.

(3-1) First Modification

A representative value of the normalized strain value corresponding to each of plurality of partial regions, into which a heart muscle is divided, can be calculated. For example, an average or median value of a plurality of normalized strain values respectively measured at the partial regions is employed as the representative value. Thus, a display method efficient in scoring at each of the regions is realized. FIG. 9 illustrates an example of such a method.

(3-2) Second Modification

The apparatus may be adapted to calculate and display numerical difference between a standard change pattern and a measured change pattern of the normalized strain value.

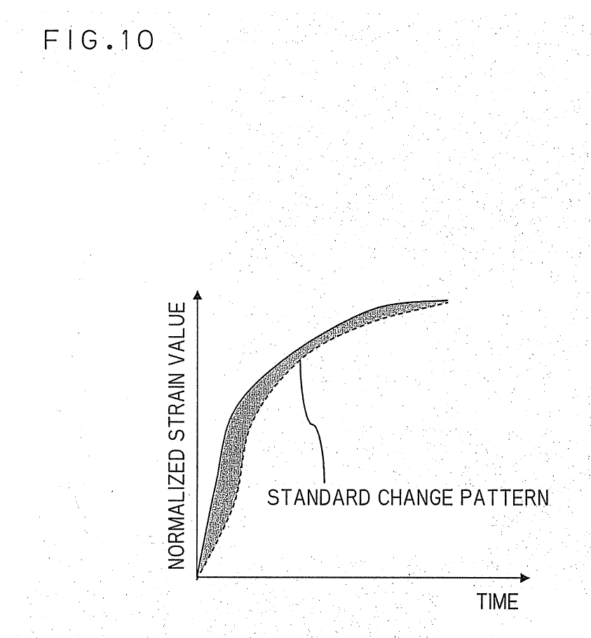

For example, as illustrated in FIG. 10, the numeric conversion of the difference between the standard change pattern and the measured change pattern of the normalized strain value can be achieved by utilizing the area of a space between the standard change pattern and the measured change pattern or by a total sum of difference values therebetween. Consequently, the difference between a case, in which the normalized strain value drastically changes, and a case, in which the normalized strain value slowly changes, can quantitatively be obtained.

(3-3) Third Modification

The time interval can optionally be set. Only a systole time may be set to be the time interval. Alternatively, only a diastole time may be set to be the time interval. Alternatively, one heartbeat period may be set to be the time interval.

The setting of only a systole time to be the set time interval is suitable for determining the rate and the timing of change of the strain value in the systole time.

Also, the setting of only a diastole time to be the set time interval is suitable for determining the rate and the timing of change of the strain value in the diastole time. In diagnosis of cardiac diseases, a delay of increase of a strain value in the systole time, and a delay in diastolic timing in the diastole time can be used as measures for diagnosis. Thus, the use of the normalized strain value facilitates the discrimination of manners of change in contraction and enlargement of a heart. Information on the normalized strain value is useful information on diagnosis.

(3-4) Fourth Modification

Although the wall thickness of a heart (the distance in the direction of thickness of a heart muscle) is employed as a measurement object, the measurement object according to the invention is not limited thereto. A distance in the direction of length of the heart muscle may be employed as a measurement object used to measure a strain value.

(4) Advantages of the Embodiments

The use of the normalized strain value in the heart function evaluation can solve the problem caused in the related evaluation based on the area of the ventricle lumen, which is obtained from the inner contour of a heart muscle. The embodiments of the present invention can solve, for example, the problems in that in a case where decrease in function of expanding and contracting heart muscles locally occurs, a decrease in motion of the endocardial border of an abnormal part pulled by the movement of neighboring normal heart muscles does not clearly occur, and that consequently, in such a case, a clear difference in the evaluation value calculated from change in the endocardial border does not occur. An evaluation value more clearly reflecting the abnormality of the heart function can be obtained.

Also, the embodiments of the present invention can solve the problems in that in the case of the related method adapted to evaluate the heart function by dividing the heart muscle into local parts, a central point for calculating the cross-sectional area of each of parts, into which the lumen is divided, is needed, and that in a case where the central point is fixed, the parallel translation of the entire heart affects the cross-sectional area of each of the parts, into which the lumen is divided, it becomes necessary to determine the central point in response to the translation of the entire heart so as to prevent the cross-sectional area of each of the parts from being affected by the translation of the entire heart. That is, in the case of using the normalized strain value, the heart muscle can be divided into local parts by being divided by, for example, a length along the heart muscle. Thus, there is no necessity for determining such a central point.

As described above, the embodiment of the present invention can provide the heart function analysis apparatus 10 enabled to obtain the evaluation value clearly reflecting the abnormality of the heart function by calculating the temporal change of the normalized strain value, and to eliminate necessity for determining the central point even in a case where the heart function is evaluated by dividing a heart muscle into local parts.

What is claimed is:

1. An apparatus for analyzing a motion of a heart by using time-series image data representing an image of the heart, the apparatus comprising:
a strain-value acquisition unit configured to acquire time-series strain values corresponding to a part of said heart according to the time-series image data;
a normalization unit configured to calculate time-series normalized strain values by normalizing the time-series strain values; and
an output unit configured to output the time-series normalized strain values.

2. The apparatus according to claim 1, further comprising:
a tracking point setting unit configured to set two tracking points at said part of said heart, which is represented by reference image data included in the time-series image data; and
a tracking unit configured to obtain positions of said two tracking points at said part represented by each piece of the time-series image data,
wherein said strain value acquisition unit acquires time-series strain values corresponding to a part of said heart according to a distance between said two tracking points at said part represented by each piece of the time-series image data.

3. The apparatus according to claim 2, wherein said tracking point setting unit sets said tracking points on an inner contour and an outer contour of a heart muscle of said heart one by one.

4. The apparatus according to claim 2, wherein said tracking point setting unit sets said two tracking points along a heart muscle of said heart.

5. The apparatus according to claim 1, wherein said normalization unit normalizes the strain value to a normalized strain value that is a measure common to a plurality of parts of said heart.

6. The apparatus according to claim 1, wherein said normalization unit defines the normalized strain value by a ratio of a maximum fluctuation range of the strain value in a time interval of analysis at said part to a difference between a current strain value and a minimum strain value.

7. The apparatus according to claim 1, wherein said normalization unit defines the normalized strain value by a ratio of a difference between an initial strain value and a terminal strain value in a time interval of analysis at said part to a difference between a current strain value and a minimum strain value.

8. The apparatus according to claim 1, further comprising:
an evaluation value calculation unit configured to calculate an evaluation value representing a difference between a temporal change pattern of the normalized strain value and a predetermined standard change pattern.

9. The apparatus according to claim 1, further comprising:
a reach time calculation unit configured to calculate a time at which the normalized value reaches a predetermined value.

10. The apparatus according to claim 6, wherein said output unit superposedly displays a color corresponding to a normalized strain value at a predetermined time in the analysis time interval on the image of said heart.

11. The apparatus according to claim 1, wherein said normalization unit calculates a representative value of the normalized strain value corresponding to each of plurality of regions into which a heart muscle is divided.

12. A method for analyzing a motion of a heart by using time-series image data representing an image of the heart, the method comprising steps of:
acquiring time-series strain values corresponding to a part of said heart according to the time-series image data;
calculating time-series normalized strain values by normalizing the time-series strain values; and
outputting the time-series normalized strain values.

13. The method according to claim 12, wherein said step of acquiring time-series strain values includes a step of obtaining the strain value from a distance in a direction of thickness or length of a heart muscle.

14. The method according to claim 12, wherein said step of calculating time-series normalized strain values includes a step of normalizing the strain value to a normalized strain value that is a measure common to a plurality of parts of said heart.

15. The method according to claim 12, wherein said step of calculating time-series normalized strain values includes a step of defining the normalized strain value by a ratio of a maximum fluctuation range of the strain value in a time interval of analysis at said part to a difference between a current strain value and a minimum strain value.

16. The method according to claim 12, wherein said step of calculating time-series normalized strain values includes a step of defining the normalized strain value by a ratio of a difference between an initial strain value and a terminal strain value in a time interval of analysis at said part to a difference between a current strain value and a minimum strain value.

17. The method according to claim 12, further comprising a step of:
calculating an evaluation value representing a difference between a temporal change pattern of the normalized strain value and a predetermined standard change pattern.

18. The method according to claim 12, further comprising a step of:
calculating a time at which the normalized value reaches a predetermined value.

19. The method according to claim 15, further comprising a step of:
superposedly displaying a color corresponding to a normalized strain value at a predetermined time in the analysis time interval on the image of said heart.

20. The method according to claim 12, further comprising a step of:
calculating a representative value of the normalized strain value corresponding to each of plurality of regions into which a heart muscle is divided.
